(19) United States
(12) Patent Application Publication
(10) Pub. No.: US 2015/0002538 A1
(43) Pub. Date: Jan. 1, 2015

(54) ULTRASOUND IMAGE DISPLAY METHOD AND APPARATUS

(71) Applicant: SAMSUNG ELECTRONICS CO., LTD., Suwon-si (KR)

(72) Inventors: Kiwon SOHN, Seoul (KR); Ho-kyung KANG, Seoul (KR); Jong-geun PARK, Seoul (KR); Yu-mi SOHN, Seoul (KR); Young-yoon LEE, Yongin-si (KR); Jin-woo YIM, Seongnam-si (KR); Min-su CHEON, Yongin-si (KR)

(73) Assignee: SAMSUNG ELECTRONICS CO., LTD., Suwon-si (KR)

(21) Appl. No.: 14/286,567

(22) Filed: May 23, 2014

(30) Foreign Application Priority Data
Jun. 26, 2013 (KR) 10-2013-0073976

(51) Int. Cl. A61B 8/08; G06T 11/60; A61B 8/00

(52) U.S. Cl. CPC A61B 8/5223; A61B 8/463; G06T 11/60
USPC 345/629

(57) ABSTRACT

An ultrasound image display method displays an ultrasound image, detects a shadow pattern for determining an internal state of an object displayed in the ultrasound image, determines the number of the detected shadow patterns, and displays the determined number of shadow patterns.

### CROSS-REFERENCE TO RELATED APPLICATION

This application claims priority from Korean Patent Application No. 10-2013-0073976, filed on Jun. 26, 2013, in the Korean Intellectual Property Office, the disclosure of which is incorporated herein in its entirety by reference.

### BACKGROUND

1. Field

One or more exemplary embodiments relate to a method and apparatus for detecting shadow patterns of ultrasound images and for displaying the detected result.

2. Description of the Related Art

An X-ray apparatus or a computed tomography (CT) apparatus is mainly used for diagnosing a lung disease, but when the X-ray apparatus or the CT apparatus is used, a human body is exposed to a large amount of radiation. Therefore, a method that diagnoses a lung disease by using ultrasound waves with no radiation exposure is being researched.

A diagnostician needs to analyze the shadow patterns generated by reverberation of ultrasound waves caused by body fluid, in order to diagnose extravascular lung water (EVLW) by using ultrasound imaging. However, the shadow patterns may be repeatedly generated and dissipated at a very high speed, and for this reason, the operator cannot easily detect the shadow patterns.

### SUMMARY

One or more exemplary embodiments include a method and apparatus that detect a shadow pattern of an ultrasound image and display the detected result, for predicting a disease by using the ultrasound image.

Additional aspects will be set forth in part in the description which follows and, in part, will be apparent from the description, or may be learned by practice of the presented embodiments.

According to one or more exemplary embodiments, an ultrasound image display method includes: displaying an ultrasound image; detecting a shadow pattern for determining an internal state of an object displayed in the ultrasound image; determining the number of the detected shadow patterns; and displaying the determined number of shadow patterns.

The displaying of an ultrasound image may include: determining a position of a probe from which the ultrasound image is acquired; and determining the number of detected shadow patterns by determined position of the probe, wherein the displaying of an ultrasound image further includes determining a prediction disease corresponding to number of shadow patterns by determined position of the probe by using a database for diseases.

The detecting of a shadow pattern may include detecting a shadow pattern of each image of an ultrasound image sequence acquired over a first time period. The displaying of the determined number may include displaying the number of detected shadow patterns in the ultrasound image sequence acquired over the first time period.

The determining of a position may include determining the position of the probe on a basis of at least one of an input value inputted by a user and a measurement value measured by a sensor included in the probe.

The detecting of a shadow pattern may include detecting a shadow pattern of each image of an ultrasound image sequence which is continued for a certain time, and the displaying of the determined number may include displaying the number of detected shadow patterns in the ultrasound image sequence which is continued for the certain time.

The shadow pattern may be a hyperechoic shadow which is displayed in a comet-tail shape from a pleura, displayed in the ultrasound image, to a lower boundary line of the ultrasound image.

According to one or more exemplary embodiments, an ultrasound image display method includes: displaying an ultrasound image; detecting a shadow pattern for determining an internal state of an object displayed in the ultrasound image; and before the detected shadow pattern is dissipated in the ultrasound image with time, displaying image sticking of the detected shadow pattern at a position, in which the detected shadow pattern displayed, for a certain time.

According to an aspect of an exemplary embodiment, it is provided an ultrasound image display method including: displaying a time-ordered sequence of ultrasound image frames; detecting one or more shadow patterns for determining an internal state of an object displayed in the time-ordered sequence of ultrasound image frames; and before a detected shadow pattern of the one or more shadow patterns disappears while displaying the time-ordered sequence of ultrasound image frames, performing image sticking of the detected shadow pattern at a position at which the detected shadow pattern was displayed before disappearing, for a time period.

The shadow pattern may be a hyperechoic shadow which is displayed in a comet-tail shape from a pleura, displayed in the ultrasound image, to a lower boundary line of the ultrasound image.

According to an aspect of an exemplary embodiment, there is provided an ultrasound image display apparatus including: a controller that detects one or more shadow patterns for determining an internal state of an object displayed in an ultrasound image, and determines the number of the detected shadow patterns; and a display that displays the ultrasound image and the determined number of shadow patterns.

The ultrasound image display apparatus may further include a database for diseases, wherein the controller determines a position of a probe from which the ultrasound image is acquired, and determines the number of detected shadow patterns by determined position of the probe.

The position of the probe may be determined on a basis of at least one of an input value inputted by a user and a measurement value measured by a sensor included in the probe.

The controller may detect a shadow pattern of each image of an ultrasound image sequence which is continued for a certain time, and the display may display the number of detected shadow patterns in the ultrasound image sequence which is continued for the certain time.

The shadow pattern may be a hyperechoic shadow which is displayed in a comet-tail shape from a pleura, displayed in the ultrasound image, to a lower boundary line of the ultrasound image.

According to an aspect of an exemplary embodiment, it is provided an ultrasound image display apparatus including: a controller that detects one or more shadow patterns for determining an internal state of an object displayed in a sequence of ultrasound images; and a display that displays the sequence of ultrasound images successively according to the acquisition time of each image, and before the detected shadow patterns disappears, as images of the sequence of ultrasound images are successively displayed, performs image sticking of the detected shadow patterns at a position at which the detected shadow pattern was displayed before disappearing, for a time period.

According to one or more exemplary embodiments, an ultrasound image display apparatus includes: a controller that detects one or more shadow patterns for determining an internal state of an object displayed in an ultrasound image; and a display that displays the ultrasound image, and before the detected shadow pattern is dissipated in the ultrasound image with time, displays image sticking of the detected shadow pattern at a position, in which the detected shadow pattern displayed, for a certain time.

The shadow pattern may be a hyperechoic shadow which is displayed in a comet-tail shape from a pleura, displayed in the ultrasound image, to a lower boundary line of the ultrasound image.

According to an aspect of an exemplary embodiment, it is provided an ultrasound image display method. The method may include receiving a time-ordered sequence of ultrasound images corresponding to ultrasound images collected from of an object over a time period; displaying the time-ordered sequence of images according to the time-order; and detecting, in the images of the sequence of ultrasound images, one or more shadow patterns for determining an internal state of an object displayed in the sequence of ultrasound images.

The time-ordered sequence of images may include a first sub-sequence of images displayed over a first time period, and a second sub-sequence of images displayed over a second time period. A first shadow pattern, of the one or more shadow patterns, may be detected in the first sub-sequence of images and is not detected in the second sub-sequence of images. The first shadow pattern may be displayed or superimposed on the images in the second sub-sequence of images.

The second time period may be of such length that an operator can properly inspect the shadow pattern. The length of the second time period is adjustable by an operator. The first time period may be so short that an operator cannot properly inspect the first shadow pattern.

The method may further include displaying, in the second sub-sequence of images, a marker associated with the first shadow pattern and indicating a position of the first shadow pattern.

The foregoing general description and the following detailed description are only exemplary and explanatory.

### BRIEF DESCRIPTION OF THE DRAWINGS

The above and/or other aspects will become more apparent and more readily appreciated from the following description of exemplary embodiments, taken in conjunction with the accompanying drawings, in which:

FIG. 1 is a flowchart illustrating an ultrasound image display method according to an exemplary embodiment;

FIG. 2 is a flowchart illustrating a method of determining a predicted disease by using an ultrasound image, according to an exemplary embodiment;

FIG. 3 is a flowchart illustrating an ultrasound image display method according to another exemplary embodiment;

FIG. 4 is a block diagram illustrating an internal structure of a display apparatus for displaying an ultrasound image, according to an exemplary embodiment;

FIG. 5 is a block diagram illustrating an internal structure of each of a display apparatus and a probe, according to an exemplary embodiment;

FIG. 6 shows diagrams illustrating an example of a shadow pattern;

FIG. 7 shows diagrams illustrating a B-line as an example of shadow patterns according to an exemplary embodiment;

FIG. 8A shows diagrams illustrating the displaying of a shadow pattern according to another exemplary embodiment;

FIG. 8B shows diagrams illustrating the displaying of a shadow pattern according to another exemplary embodiment;

FIG. 9 is a diagram illustrating the displaying of a number of shadow patterns according to an exemplary embodiment;

FIG. 10 is a diagram illustrating body parts of an object enabling an ultrasound image to be acquired, according to an exemplary embodiment; and

FIG. 11 is a diagram showing an example of a diagnosis table according to an exemplary embodiment.

### DETAILED DESCRIPTION

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

Reference will now be made in detail to exemplary embodiments, examples of which are illustrated in the accompanying drawings, wherein like reference numerals refer to like elements throughout. In this regard, exemplary embodiments may have different forms and should not be construed as being limited to the descriptions set forth herein. Accordingly, the exemplary embodiments are merely described below, by referring to the figures, to explain aspects of the present description. Expressions such as “at least one of,” when preceding a list of elements, modify the entire list of elements and do not modify the individual elements of the list.

The terms or words used in the present specification and claims, are not limited to common or dictionary meaning and should be interpreted based on the meanings and concepts corresponding to technical aspects of the present teaching as defined on the basis of the principle. Therefore, the configuration shown in the drawings embodiments described herein, as in effect at the time of this application, and it should be understood that various equivalents, and a modified example can be implemented.

Although some features may be described with respect to individual exemplary embodiments, aspects need not be limited thereto such that features from one or more exemplary embodiments may be combinable with other features from one or more exemplary embodiments.

The aspects of exemplary embodiments are not limited to the disclosed operations and sequence of operations. For instance, operations may be performed by various elements and components, may be consolidated, may be omitted, and may be altered without departing from the spirit and scope of the present teaching.

Hereinafter, exemplary embodiments will be described in detail with reference to the accompanying drawings.

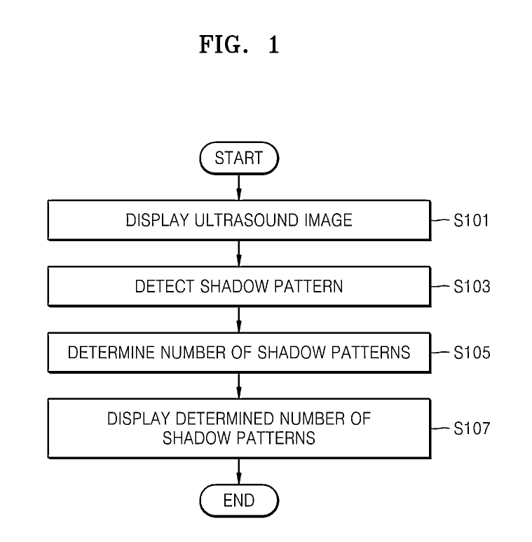

FIG. 1 is a flowchart illustrating an ultrasound image display method according to an exemplary embodiment.

Referring to FIG. 1, an ultrasound image display apparatus according to an exemplary embodiment may display an ultrasound image in operation S101.

The ultrasound image display apparatus may be connected to a probe, and may receive an ultrasound image from the probe or a device storing an ultrasound image, thereby displaying the received ultrasound image. Alternatively, the ultrasound image display apparatus may display an ultrasound image stored therein. The probe may collect a sequence of images over a certain period of time. The sequence of images may be stored as a time-ordered sequence of images according to the time at which they have been collected. The time-ordered sequence of images may be arranged as image frames in a video which may be played or stored as a video file. The ultrasound image display apparatus may receive the time-ordered sequence of images from the probe or from a storage device such as a computer memory. The ultrasound image display apparatus may display the time-ordered sequence of images or video as video frames. This way the operator can visualize the evolution in time of the ultrasound images collected by the probe. The ultrasound image display apparatus may display the ultrasound images in “real time” or from a memory storage.

Therefore, an ultrasound image or video, which is acquired by the probe in real time, may be displayed, or a pre-stored ultrasound image or video may be reproduced and displayed by the ultrasound image display apparatus.

In operation S103, the ultrasound image display apparatus may detect a shadow pattern, used to determine an internal state of an object, in the displayed ultrasound images of a sequence of images. In an exemplary embodiment, the ultrasound image display apparatus may detect the shadow pattern at each time of a temporally continued ultrasound image. For example, the ultrasound image display apparatus may detect a sequence of images including N image frames. The images in the sequence may be collected at a plurality of times T1 to TN by an ultrasound probe from a specific part of an object. The times T1 to Tn may be spaced in time by a frame time. The ultrasound apparatus may detect the shadow patterns in each of the N image frames.

At this time, the ultrasound image display apparatus may analyze the ultrasound image and detect a shadow pattern having a shape for detecting a certain structural feature of the object investigated or a disease by using the analyzed result. The ultrasound apparatus may analyze individual image frames of an acquired sequence of images and detect shadow patterns having certain characteristics consistent with certain structural features of the object investigated or a disease. For example, the ultrasound image display apparatus may analyze the ultrasound image by using a contrast value of the ultrasound image. Alternatively, the ultrasound image display apparatus may detect an edge region to analyze the ultrasound image. However, the analysis of the ultrasound image according to an exemplary embodiment is not limited thereto, and the ultrasound image may be analyzed by various methods. For example, various image recognition methods and software may be employed to analyze the image frames and detect specific shadow patterns. Various model shadow patterns and the structural features they correspond to may be stored in a data base for comparison with the patterns in the acquired ultrasound images.

In an exemplary embodiment the shadow pattern may be for diagnosing extravascular lung water (EVLW) among lung diseases, and may be a B-line that is a hyperechoic shadow traveling from a pleura to an end of the ultrasound image. When ultrasound waves pass through water, an intensity of the ultrasound waves is not reduced, and thus, the ultrasound waves are displayed as a hyperechoic shadow compared to other parts. Therefore, when body fluids are accumulated in the lungs, the B-line that is a virtual image may be observed in ultrasound images obtained by imaging the lungs.

An A-line, which is shown as a bright horizontal line, may be observed near the pleura in an ultrasound image acquired from normal lungs, but when the B-line is observed in an ultrasound image because body fluids are accumulated in the lungs, the A-line, which is shown in normal lungs, is not observed.

A diagnostician needs to analyze the number of B-lines of an ultrasound image, for diagnosing EVLW. However, B-lines may be repeatedly generated and dissipated at a very high speed, for example, at every about 0.1 sec, and for this reason, it is difficult to recognize the B-line with the eyes. For example, the sequence of images collected by the probe may be displayed at a rate of, for example, 50 frames per second (i.e. 20 ms per frame) over a 20 second period (i.e. 1000 frames in 20 seconds). A certain B-line may be displayed, for example, in frames 23-27, 100-105, 240-250, and 860-870. Thus, the B-line may be displayed only for a fraction of a second in the 20 seconds. That means that the B-line may appear in frame 23 and disappear after 80 ms at frame 27, then B-line appears again for a fraction of a second in the 20 second period. An operator or diagnostician visualizing the sequence of images displayed over the 20 seconds cannot recognize or properly inspect the B-line within such short periods of time (e.g. the 80 ms between frames 23-27).

According to an exemplary embodiment, the ultrasound image display apparatus may automatically detect a shadow pattern which is quickly changed like the B-line, determine the number of shadow patterns, and display the determined number of shadow patterns in order for a diagnostician to determine the number of shadow patterns. Therefore, the diagnostician can determine the number of shadow patterns at a degree of accuracy that is higher than a case of recognizing the shadow patterns with the eyes to determine the number of shadow patterns.

For example, in a sequence of image frames displayed by the ultrasound image display apparatus (e.g. the 1000 images detected over a 20 second period as presented in the previous paragraph), the apparatus may automatically detect a first shadow pattern in a first sub-sequence of images (e.g. frames 23-27) and a second shadow pattern in a second sub-sequence of images (e.g. frames 450-462). Then, right after detection the first shadow pattern in the first sub-sequence of images (i.e. frames 23-27) the apparatus may display the first shadow pattern in subsequently displayed frames (e.g. frames 28-1000) by, for example, superimposing the first shadow pattern or performing image sticking of the second shadow pattern over the images of frames 28-1000 at a position corresponding to the position of the first shadow pattern. Similarly, right after detection the second shadow pattern in the second sub-sequence of images (i.e. frames 450-462) the apparatus may display the second shadow pattern in subsequently displayed frames (e.g. frames 462-1000) by, for example, superimposing the second shadow pattern or performing image sticking of the second shadow pattern over the images of frames 462-1000 at a position corresponding to the position of the second shadow pattern. Thus, the shadow patterns may be displayed for at least a couple of seconds (e.g. 2 seconds or more, or about 5 seconds) such that an operator can properly inspect the shadow patterns. The display time may be adjusted according to the need of the operator. If the shadow patterns are displayed for less than a second an operator cannot properly inspect the shadow pattern.

Further, in an exemplary embodiment the shadow patterns may be first detected upon acquiring the sequence of images and displaying them (e.g. as a video including 1000 frames) in “real time”. Then, upon replaying the sequence of frames previously acquired, the shadow patterns may be displayed in each of the frames of the video (e.g. in each of the 1000 frames).

Here, the shadow pattern is not limited to the B-line for diagnosing EVLW, and may be a shadow pattern that is repeatedly generated and dissipated in a continuous ultrasound image sequence.

In operation S105, the ultrasound image display apparatus may determine the number of shadow patterns that are detected in operation S103. The number of shadow patterns may be determined as the number of shadow patterns which are detected from an ultrasound image displayed at a current time, or may be determined as the total number of shadow patterns detected from each image of an ultrasound image sequence which is acquired for a certain period of time. Alternatively, the number of shadow patterns may be an average value of a total of shadow patterns detected from each image of an ultrasound image sequence which is continued for a certain time at least two times or more. In an exemplary embodiment an image recognition method may be used to identify specific shadow patterns detected in the sequence of frames and to distinguish between different shadow patterns. For example, in a 1000 frames sequence, the apparatus may detect shadow patterns in the frame sub-sequences 120-130, 340-350, and 609-620. Upon analyzing the shadow patterns detected in these frames, the apparatus may determine that the shadow pattern in frame sub-sequence 120-130 is the same as the one in frame subsequence 609-620, and that the shadow pattern in frame sub-sequence 340-350 is different from the shadow pattern in frames 120-130 and 609-620. Thus, the apparatus may determine that two different types of shadow patterns are displayed in the 1000 frames sequence.

A certain time, for which the number of shadow patterns is detected, may be determined according to a user input or a predetermined setting value. For example, an user may set the apparatus to display a shadow pattern for 10 seconds after the shadow pattern has been detected in an image frame.

In operation S107, the ultrasound image display apparatus may display the number of shadow patterns which is determined in operation S105. The ultrasound image display apparatus may display at least one of the number of shadow patterns (which are detected from an ultrasound image displayed at a current time) and the total number of shadow patterns detected from each image of an ultrasound image sequence which is continued for a certain time. The number of shadow patterns displayed in operation S107 may be referred to by a diagnostician in diagnosing a disease of an object.

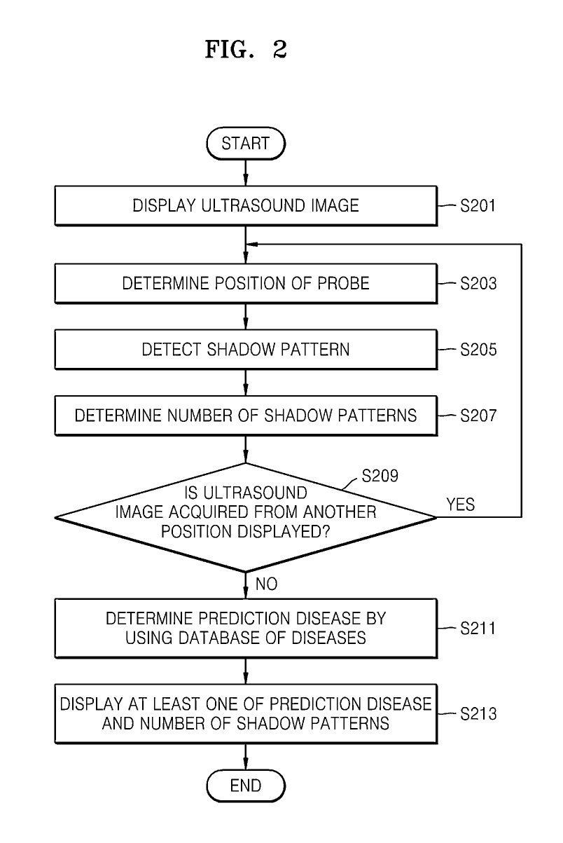

FIG. 2 is a flowchart illustrating a method of determining a predicted disease by using an ultrasound image, according to an exemplary embodiment. Operations S201, S205 and S207 of FIG. 2 respectively correspond to operations S101, S103 and S105 of FIG. 1, and thus, their detailed description is not provided.

Referring to FIG. 2, the ultrasound image display apparatus according to an exemplary embodiment may display an ultrasound image, a time-ordered sequence of images, or a video in operation S201.

In operation S203, the ultrasound image display apparatus may determine a position of a probe from which the displayed ultrasound image the time-ordered sequence of images or the video is acquired. The position of the probe may be determined by using a magnetic field sensor (included in the probe) for determining a position; however, the position of the probe may be determined by various methods without being limited thereto.

In operation S205, the ultrasound image display apparatus may detect a shadow pattern, used to determine an internal state of an object, from the displayed ultrasound image, or sequence of images or video. The detected shadow pattern may be used to predict a disease.

In operation S207, the ultrasound image display apparatus may determine the number of shadow patterns which have been detected in operation S205.

In operation S209, when the ultrasound image display apparatus displays an ultrasound image or video acquired from another position or the position of the probe is changed, the ultrasound image display apparatus may repeat operation S203 of determining the position of the probe, operation S205 of detecting the shadow pattern, and operation S207 of determining the number of shadow patterns. Therefore, the ultrasound image display apparatus may detect a shadow pattern by using ultrasound images or videos respectively detected from different body parts of the object, and determine the number of detected shadow patterns. The ultrasound image display apparatus may detect shadow patterns by using ultrasound images or videos respectively detected from a same body part but from different positions of the probe with respect to the body part. According to an exemplary embodiment, the ultrasound image display apparatus may receive position information from the magnetic field sensor of the probe in real time and determine the number of shadow patterns detected from the respective positions.

In operation S211, the ultrasound image display apparatus may determine a predicted disease of the object by using a database of diseases. The database of diseases may include information on a disease of the object which is predictable on the basis of the number of detected shadow patterns by body part of the object. That is, the database of diseases may include information on a disease of the object corresponding to the number of detected shadow patterns by body part of the object.

Therefore, according to an exemplary embodiment, the ultrasound image display apparatus may determine a predicted disease of the object on the basis of the position of the probe which is determined in operation S203 and the number of shadow patterns which has been determined for the respective positions of the probe in operation S207.

Examples of predictable diseases in an exemplary embodiment include pulmonary edema, acute respiratory distress syndrome (ARDS), pneumothorax, acute coronary syndrome, and pulmonary fibrosis.

In operation S213, the ultrasound image display apparatus may display at least one of the predicted disease (which has been determined in operation S211) and the number of shadow patterns. That is, the ultrasound image display apparatus may display at least one of the number of shadow patterns detected from a currently displayed ultrasound image, a diagnosis table that shows the number of detected shadow patterns by body part of the object, and the predicted disease which has been determined in operation S211. The number of detected shadow patterns corresponding to a body part of the object may be the number of shadow patterns which are detected during a certain period of time in which ultrasound imaging of the body part is performed.

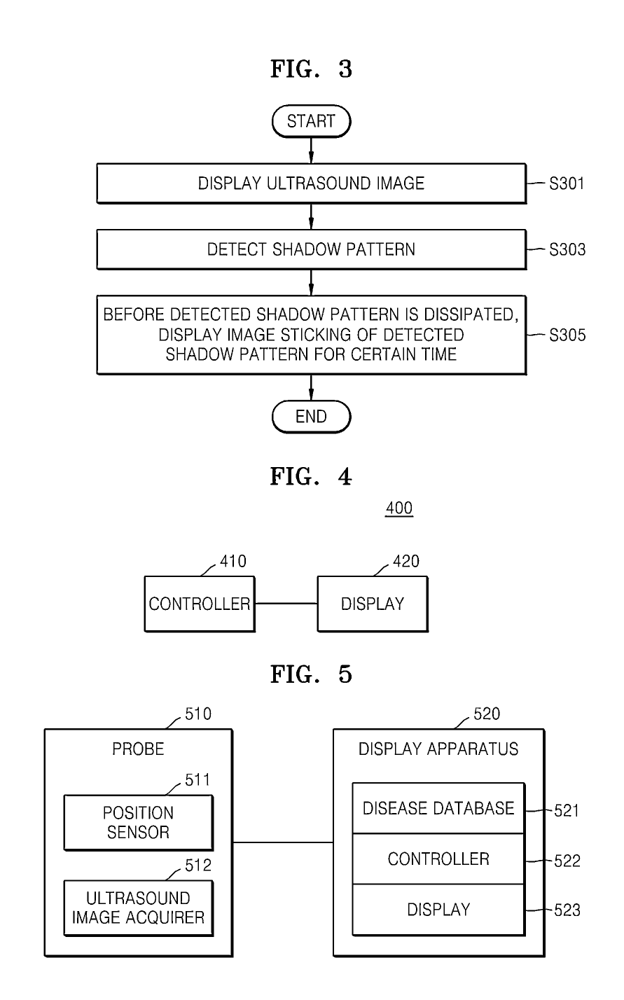

FIG. 3 is a flowchart illustrating an ultrasound image display method according to another exemplary embodiment. Operations S301 and S303 of FIG. 3 respectively correspond to operations S201 and S205 of FIG. 2, and thus, their detailed description is not provided.

Referring to FIG. 3, the ultrasound image display apparatus may display an ultrasound image in operation S301.

In operation S303, the ultrasound image display apparatus may detect a shadow pattern, used to determine an internal state of an object, from the displayed ultrasound image. The detected shadow pattern may be referred to for a diagnostician predicting a disease.

In operation S305, the ultrasound image display apparatus may perform image sticking of the detected shadow pattern at a position, at which the shadow pattern is displayed, for a certain time before the detected shadow pattern is generated in and dissipated from the ultrasound image. In another exemplary embodiment, the image sticking of the shadow pattern may be displayed in the same shape as that of the displayed shadow pattern, or may be displayed by the degree in which the diagnostician can recognize the image sticking as the shadow pattern. For example, when the shadow pattern is the B-line usable for diagnosing a lung disease, a marker having a straight-line shape or a rectangular shape may be displayed at a position with the detected shadow pattern thereat. The marker may be superimposed over the shadow pattern. The marker or the image sticking may be displayed for a certain time (which enables the diagnostician to easily recognize the marker or image sticking) even after the shadow pattern is dissipated, and then may be gradually dissipated.

According to another exemplary embodiment, the ultrasound image display apparatus may display a marker or perform image sticking of a shadow pattern, which is repeatedly generated and dissipated for a short time, for a certain time which enables a diagnostician to recognize the marker or image sticking, and thus, the diagnostician can easily recognize generation of the shadow pattern. Accordingly, the diagnostician can diagnose diseases of a diagnosable object according to whether a shadow pattern is generated, with reference to a marker or image sticking of the shadow pattern displayed for a certain time.

An internal structure of the above-described ultrasound image display apparatus will now be described in detail with reference to FIGS. 4 and 5.

FIG. 4 is a block diagram illustrating an internal structure of an ultrasound image display apparatus 400 according to an exemplary embodiment.

Referring to FIG. 4, the ultrasound image display apparatus 400 according to an exemplary embodiment may include a controller 410 and a display 420.

The controller 410 may detect a shadow pattern that is referred to for diagnosing a disease of an object displayed in an ultrasound image which is displayed by the display 420, and determine the number of detected shadow patterns. In an exemplary embodiment, the controller 410 may detect a shadow pattern at each time of a temporally continued ultrasound image or from a currently displayed ultrasound image. At this time, the controller 410 may perform an ultrasound image analysis, such as detection of a contrast value or edge region of the ultrasound image, and detect a shadow pattern having a shape for detecting by using the analyzed result.

The display 420 may display the ultrasound image, and display the number of shadow patterns determined by the controller 410.

At this time, the ultrasound image display apparatus 400 may be connected to a probe (not shown), and may receive an ultrasound image from the probe or a device storing an ultrasound image, thereby displaying the received ultrasound image. Alternatively, the ultrasound image display apparatus 400 may display an ultrasound image stored in a memory of the ultrasound image display apparatus 400. Therefore, an ultrasound image, which is acquired by the probe in real time, may be displayed, or a pre-stored ultrasound image may be reproduced and displayed by the display 420 of the ultrasound image display apparatus 400.

Further, according to another exemplary embodiment, the shadow pattern detected by the controller 410 is generated in the ultrasound image, and then, before the shadow pattern is dissipated, the display 420 may display a marker or image sticking of a shadow pattern, detected for a certain time, at a position with the shadow pattern displayed thereat.

FIG. 5 is a block diagram illustrating an internal structure of each of a display apparatus and a probe 510 according to an exemplary embodiment. The display apparatus 520, a controller 522, and a display 523 of FIG. 5 respectively correspond to the display apparatus 420, the controller 410, and the display 420 of FIG. 4, and thus, their detailed description is not provided.

Referring to FIG. 5, the probe 510 according to an exemplary embodiment may include a position sensor 511 and an ultrasound image acquirer 512. Also, the probe 510 may be connected to the display apparatus 520, and may transfer at least one of an ultrasound image acquired by the probe 510 and position information corresponding to each ultrasound image to the display apparatus 520.

The position sensor 511 may sense a position of the probe 510 to determine a position of the probe 510 from which the ultrasound image is acquired. The position sensor 511 may be a magnetic field sensor that senses a magnetic field generated near the probe 510 to acquire position information. Further, a position of the object and of a body part intended to be investigated by ultrasound imaging may be determined with respect to the ultrasound probe. The position of the object and of a body part may be determined, by various means, in advance or at the time the ultrasound imaging is performed.

The ultrasound image acquirer 512 may irradiate ultrasound waves on a region of interest (ROI) of an object, and receive an ultrasound echo generated by the irradiated ultrasound waves. The ultrasound image acquirer 512 may acquire an ultrasound image of the ROI by using the received ultrasound echo. The ultrasound image acquired by the ultrasound image acquirer 512 may be transferred to the display apparatus 520, and displayed by the display 523.

The display apparatus 520 according to an exemplary embodiment may include a disease database 521, the controller 522, and the display 523.

The disease database 521 may include information on diseases which are predictable on the basis of the number of detected shadow patterns by body part of an object. That is, the disease database 521 may include information on a disease of the object corresponding to the number of detected shadow patterns by body part of the object.

Generally, a diagnostician may diagnose a lung disease of an object on the basis of the number of B-lines detected from each body part of the object. That is, the B-lines may be based on diagnosis of a lung disease which causes EVLW among lung diseases.

In an exemplary embodiment, the display apparatus 520 may determine the number of B-lines detected from an ultrasound image, and determine a lung disease which is predicted by using the determined number of B-lines, a body part (i.e., position information of the probe with respect to body parts of the object) of an object from which the ultrasound image is acquired, and the disease database 521. Accordingly, the diagnostician can diagnose a disease of the object with reference to a predicted disease displayed by the display apparatus 520.

The controller 522 may detect a shadow pattern for determining an internal state of the object displayed in an ultrasound image, which is displayed by the display 523, and determine the number of detected shadow patterns. Also, the controller 522 may determine a predicted disease of the object by using the disease database, the determined number of shadow patterns, and position information of the probe with respect to the object from which the ultrasound images are acquired.

The display 523 may display the ultrasound image, and display the number of shadow patterns determined by the controller 522. Also, the controller 522 may display the predicted disease of the object determined by the controller 522 and the diagnosis table. The diagnosis table is a table that shows the number of detected shadow patterns by each body part of the object.

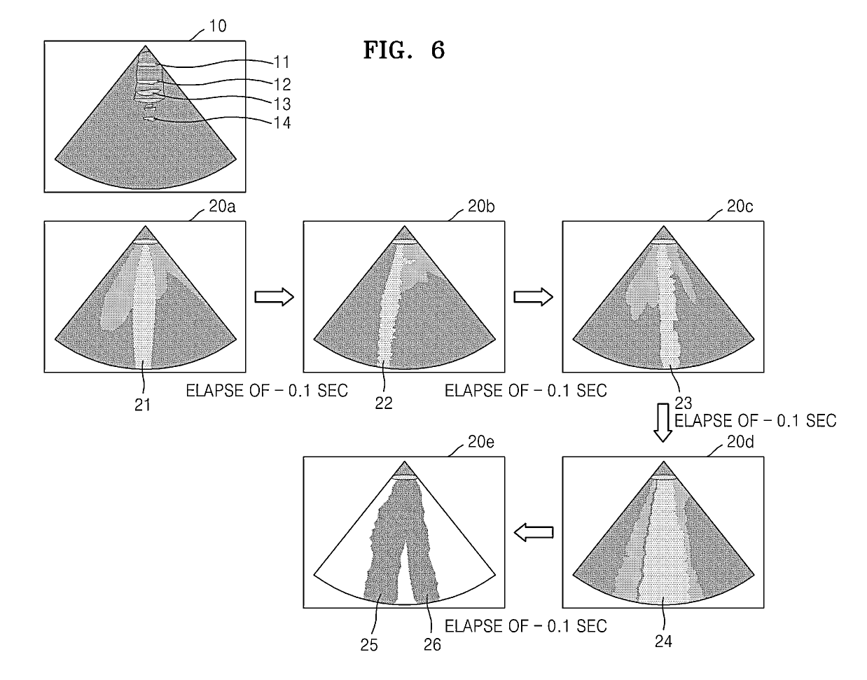

FIG. 6 is diagrams illustrating an example of shadow patterns.

Referring to FIG. 6, an example of shadow patterns, an A-line and a B-line are shown in respective shadow images.

Referring to an ultrasound image 10, A-lines 11 to 14 may be observed as bright horizontal lines indicating a lung boundary region, namely, a pleura. The A-lines are observed in an ultrasound image, which is obtained by photographing normal lungs.

Referring to an ultrasound image 20a, a B-line generated by body fluid which is in the lungs is observed. The B-line may be repeatedly generated and dissipated at intervals of 0.1 sec when the probe acquires an ultrasound image from the same body part of an object. The B-line is generated and dissipated in each of ultrasound images 20a to 20e for a very short time, and thus, it is difficult for a diagnostician to determine the number of B-lines 21 to 26 generated in each ultrasound image.

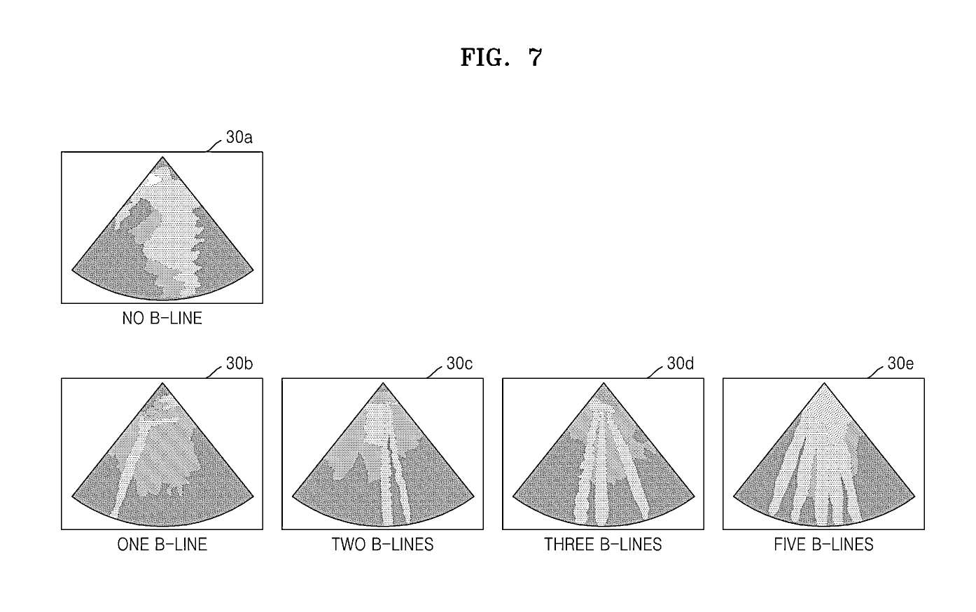

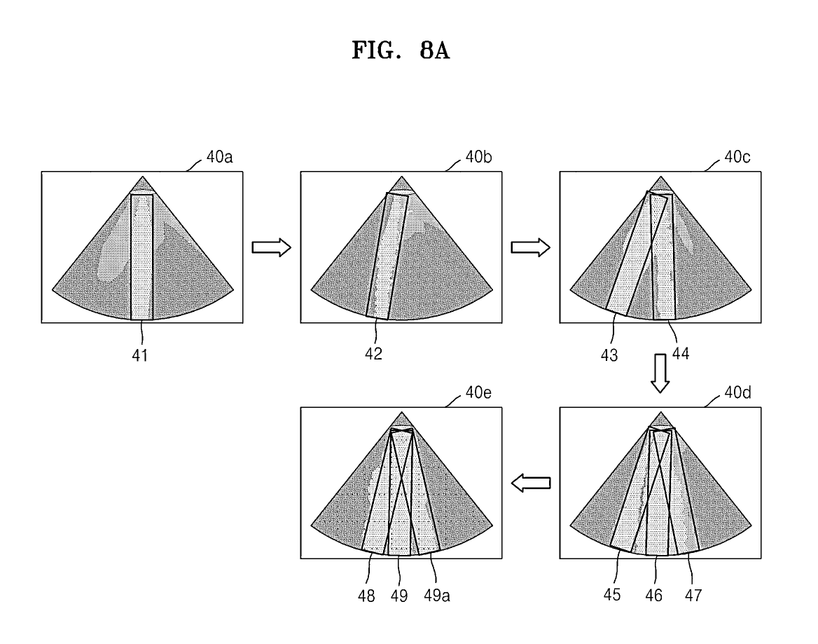

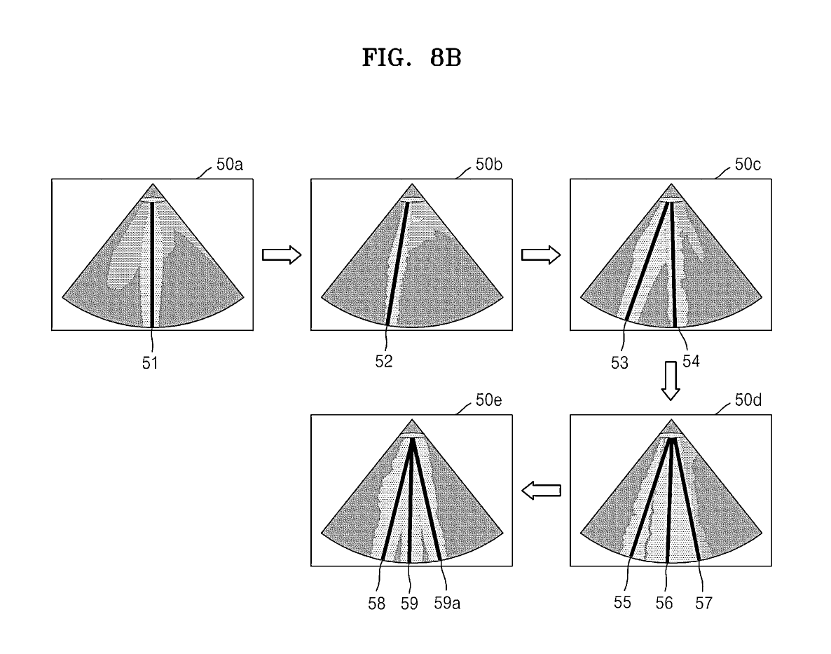

To solve such problems, embodiments of shadow patterns which are detected and displayed by the display apparatuses 400 and 520 will now be described with reference to FIGS. 7 and 8.

FIG. 7 shows diagrams illustrating a B-line as an example of shadow patterns according to an exemplary embodiment.

Referring to FIG. 7, the B-line is a hyperechoic shadow which is displayed in a tail shape from a pleura to a lower boundary line of an ultrasound image, and may be displayed in the ultrasound image. As in ultrasound images 30a to 30e of FIG. 7, one or more B-lines are observed in each of the ultrasound images 30a to 30e. The ultrasound image 30a is an image in a state in which a B-line is not shown, and the ultrasound images 30b to 30e are images in which the B-lines are shown as one to five, respectively.

FIG. 8A shows diagrams illustrating the displaying of a shadow pattern according to another exemplary embodiment. FIG. 8B shows diagrams illustrating the displaying of a shadow pattern according to another exemplary embodiment.

Referring to FIG. 8A, a plurality of detected shadow patterns may be displayed in a rectangular shape 41 to 49a in a plurality of ultrasound images 40a to 40e. Referring to FIG. 8B, a plurality of detected shadow patterns may be displayed as straight lines 51 to 59a in a plurality of ultrasound images 50a to 50e. Therefore, according to another exemplary embodiment, a diagnostician can easily recognize a shadow pattern shown in an ultrasound image by using the marker of the shadow pattern displayed by the display apparatuses 400 and 520.

Moreover, in another exemplary embodiment, the display apparatuses 400 and 520 may display the image sticking of the shadow pattern, instead of the marker of the shadow pattern, for a certain time, thereby displaying the detected shadow pattern so as to be easily recognized.

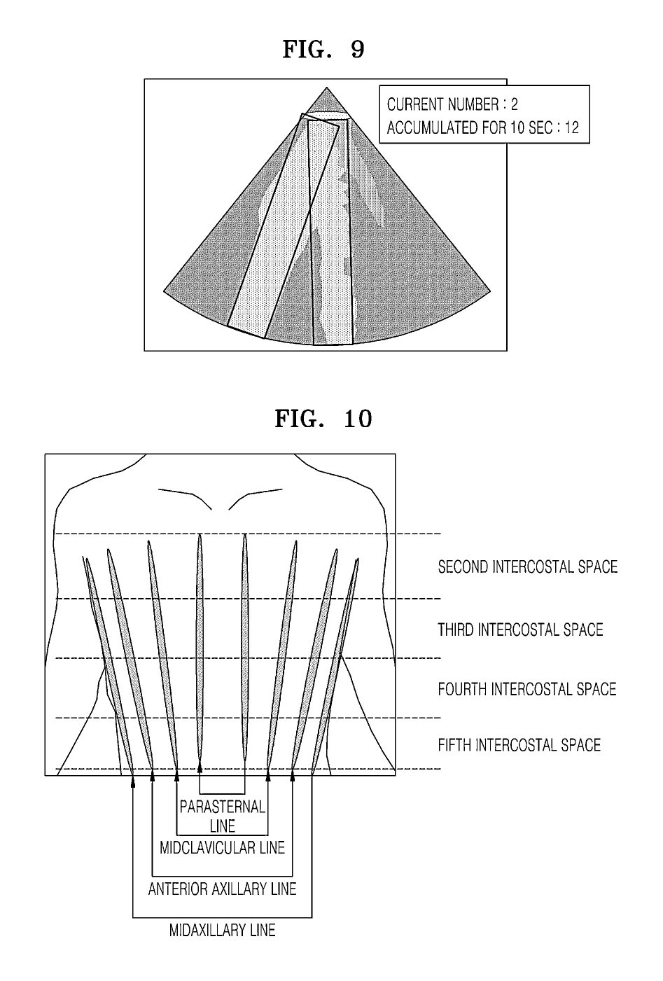

FIG. 9 is a diagram illustrating an example for displaying the number of shadow patterns according to an exemplary embodiment.

Referring to FIG. 9, the display apparatuses 400 and 520 may display at least one of the number of shadow patterns, which are detected from an ultrasound image displayed at a current time, and the total number of shadow patterns detected from each image of an ultrasound image sequence which is continued for a certain time. The number of shadow patterns which are detected from the ultrasound image displayed at a current time is two, and thus, the number of detected shadow patterns is displayed together in an ultrasound image of FIG. 9. The number of detected shadow patterns may be displayed along with the ultrasound image. A certain time, for which the number of shadow patterns is detected, may be determined according to a user input or a predetermined setting value.

FIG. 10 is an exemplary diagram illustrating body parts of an object enabling an ultrasound image to be acquired, according to an exemplary embodiment.

Referring to FIG. 10, an ultrasound image according to an exemplary embodiment may be acquired from twenty-eight body parts except a left and lowermost body part of an object. The object may be divided into 28 body parts, including second to fifth intercostal spaces, a parasternal line, a midclavicular line, an anterior axillary line, and a midaxillary line. A detection body part of an object illustrated in FIG. 10 is divided for a human, and when an object is a living thing other than a human, a detection body part may be divided differently from the human. Since a stomach is in four left and lowermost body parts, in an exemplary embodiment, the four body parts may be excluded from a diagnosis region in an ultrasound diagnosis for predicting a lung disease. The probe 510 according to an exemplary embodiment may acquire an ultrasound image of each body part of an object and position information of each ultrasound image to transfer the ultrasound image and position information to the display apparatuses 400 and 520. The display apparatuses 400 and 520 may display both the ultrasound image and the position information in order for a diagnostician to diagnose a disease of the object in consideration of a position (i.e., a body part of the object) from which the ultrasound image is acquired. Also, the display apparatuses 400 and 520 may predict the disease of the object by using the disease database, information on the position from which the ultrasound image is acquired, and the determined number of shadow patterns, and may display the predicted disease.

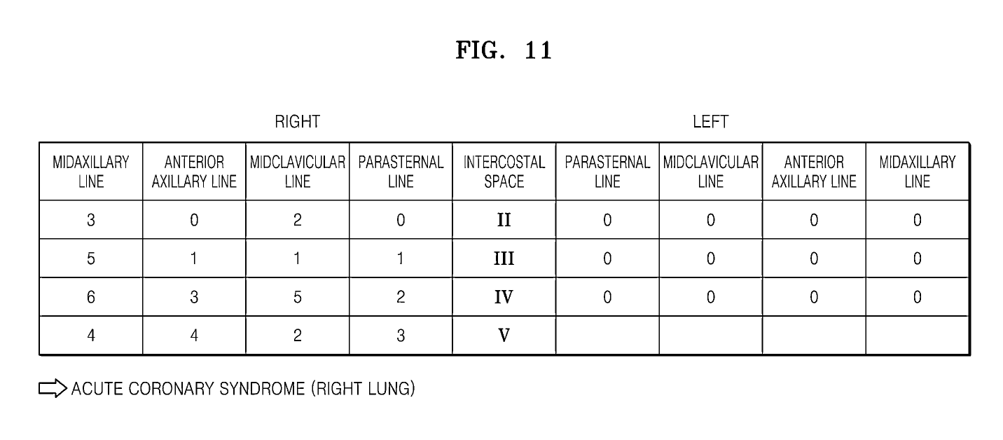

FIG. 11 is a diagram showing an example of a diagnosis table according to an exemplary embodiment.

Referring to FIG. 11, the display apparatuses 400 and 520 may display the diagnosis table that shows the number of detected shadow patterns and information on a position from which an ultrasound image with the shadow pattern detected therefrom is acquired. The diagnosis table according to an exemplary embodiment may show the number of detected shadow patterns by body part of an object. The number of shadow patterns listed in the diagnosis table may be the number of shadow patterns which are detected from an ultrasound image for a certain time, or may be an average number value of shadow patterns which are detected at least two times or more for a certain time. The diagnosis table may be used in order for a diagnostician to diagnose a predicted disease with reference to the diagnosis table.

In addition, the display apparatuses 400 and 520 may determine a predicted disease by using the diagnosis table, the number of detected shadow patterns, information on a position from which an ultrasound image with the shadow pattern detected therefrom is acquired, and the disease database, and may display the determined disease. Information on a disease which is predicted and displayed by the display apparatuses 400 and 520 may be used in order for a diagnostician to diagnose a disease of an object with reference to the disease information.

Referring to the diagnosis table of FIG. 11, the display apparatuses 400 and 520 may predict a disease of an object by using the number of B-lines detected from a right body part of the object and a detection body part, and display acute coronary syndrome being detected from a right lung part as the predicted result. Detection body parts for shadow patterns listed in the diagnosis table of FIG. 11 are divided for a human. When the display apparatuses 400 and 520 predict a disease of an object other than a human, detection body parts of the object may be set differently from the human, and the diagnosis table may be shown according to the set result. Since a stomach is in the fifth intercostal space among left body parts of the diagnosis table, when predicting a lung disease, the display apparatuses 400 and 520 may not detect a shadow pattern from an ultrasound image of the fifth intercostal space.

According to an exemplary embodiment, the display apparatus detects the number of shadow patterns of an ultrasound image which are generated and dissipated at a high speed, and displays the number of shadow patterns. Accordingly, a diagnostician can easily determine the number of shadow patterns necessary to diagnose a disease of an object.

According to another exemplary embodiment, the display apparatus displays image sticking of a detected shadow pattern, and thus, a diagnostician can easily recognize a shadow pattern of an ultrasound image which is generated and dissipated at a high speed.

According to an exemplary embodiment, a diagnostician can easily diagnose a disease of an object with reference to the diagnosis table and a disease predicted by the display apparatus.

Another exemplary embodiment can also be embodied as computer-readable codes on a computer-readable recording medium (including all devices having an information processing function). The computer-readable recording medium is any data storage device that can store data which can be thereafter read by a computer system. Examples of the computer-readable recording medium include read-only memory (ROM), random-access memory (RAM), CD-ROMs, magnetic tapes, floppy disks, and optical data storage devices.

It should be understood that the exemplary embodiments described therein should be considered in a descriptive sense only and not for purposes of limitation. Descriptions of features or aspects within each embodiment should typically be considered as available for other similar features or aspects in other embodiments. Therefore, it is intended that the present disclosure not be limited to the particular exemplary embodiments disclosed as the best mode contemplated for carrying out the present disclosure, but that the present disclosure will include all embodiments falling within the scope of the appended claims.

While only few exemplary embodiments have been described, it will be understood by those of ordinary skill in the art that various changes in form and details may be made therein without departing from the spirit and scope of the present teaching as defined by the following claims.

What is claimed is:

1. An ultrasound image display method comprising:
displaying an ultrasound image;
detecting one or more shadow patterns for determining an internal state of an object displayed in the ultrasound image;
determining a number of the detected one or more shadow patterns; and
displaying the determined number of the detected one or more shadow patterns.

2. The ultrasound image display method of claim 1, wherein the displaying of an ultrasound image comprises:
determining a position of a probe from which the ultrasound image is acquired; and
determining the number of detected one or more shadow patterns at the determined position of the probe,
wherein the displaying of the ultrasound image further comprises determining a predicted disease corresponding to the number of detected one or more shadow patterns at the determined position of the probe by using a database of diseases.

3. The ultrasound image display method of claim 2, wherein the determining of a position of the probe comprises:
determining the position of the probe based on at least one of an input value inputted by a user and a measurement value measured by a sensor included in the probe.

4. The ultrasound image display method of claim 1, wherein:
the detecting of the one or more shadow patterns comprises detecting shadow patterns of images of an ultrasound image sequence acquired over a first time period, and
the displaying of the determined number of the detected one or more shadow patterns comprises displaying the number of detected one or more shadow patterns in the ultrasound image sequence acquired over the first time period.

5. The ultrasound image display method of claim 1, wherein the detected one or more shadow patterns are hyperechoic shadows which are displayed in a comet-tail shape from a pleura, displayed in the ultrasound image, to a lower boundary line of the ultrasound image.

6. An ultrasound image display method comprising:
displaying a time-ordered sequence of ultrasound image frames;
detecting one or more shadow patterns for determining an internal state of an object displayed in the time-ordered sequence of ultrasound image frames; and
before a detected shadow pattern of the one or more shadow patterns disappears during the displaying of the time-ordered sequence of ultrasound image frames, performing image sticking of the detected shadow pattern at a position at which the detected shadow pattern was displayed before disappearing, the image sticking being performed for a predetermined time period.

7. The ultrasound image display method of claim 6, wherein the detected shadow pattern is a hyperechoic shadow which is displayed in a comet-tail shape from a pleura, displayed in the ultrasound image, to a lower boundary line of the ultrasound image.

8. An ultrasound image display apparatus comprising:
a controller configured to detect one or more shadow patterns for determining an internal state of an object displayed in an ultrasound image and determine a number of the detected one or more shadow patterns; and
a display configured to display the ultrasound image and the determined number of the one or more shadow patterns.

9. The ultrasound image display apparatus of claim 8, further comprising a database of diseases,
wherein the controller is configured to determine a position of a probe from which the ultrasound image is acquired and to determine the number of detected one or more shadow patterns at the determined position of the probe.

10. The ultrasound image display apparatus of claim 9, wherein the determined position of the probe is determined based on at least one of an input value inputted by a user and a measurement value measured by a sensor included in the probe.

11. The ultrasound image display apparatus of claim 8, wherein:
the controller is configured to determine shadow patterns of images of an ultrasound image sequence acquired over a first time period, and
the display is configured to display the number of detected one or more shadow patterns in the ultrasound image sequence acquired over the first time period.

12. The ultrasound image display apparatus of claim 8, wherein the detected one or more shadow patterns are hyperechoic shadows which are displayed in a comet-tail shape from a pleura, displayed in the ultrasound image, to a lower boundary line of the ultrasound image.

13. An ultrasound image display apparatus comprising:
a controller configured to detect one or more shadow patterns for determining an internal state of an object displayed in a sequence of ultrasound images; and
a display configured to display the sequence of ultrasound images successively according to the acquisition time of each image of the sequence of ultrasound images and before the detected one or more shadow patterns disappear, during the successive displaying of the images of the sequence of ultrasound images, the display performs image sticking of the detected shadow patterns at a position at which the detected shadow pattern was displayed before disappearing, the image sticking being performed for a predetermined time period.

14. The ultrasound image display apparatus of claim 13, wherein the detected one or more shadow patterns are hyperechoic shadows which are displayed in a comet-tail shape from a pleura, displayed in the ultrasound image, to a lower boundary line of the ultrasound image.

15. A non-transitory computer-readable storage medium storing a program for executing the method of claim 1.

16. An ultrasound image display method comprising:
receiving a time-ordered sequence of ultrasound images corresponding to ultrasound images collected from of an object over a time period;
displaying the time-ordered sequence of images according to a time-order;
detecting, in images of the sequence of ultrasound images, one or more shadow patterns for determining an internal state of an object displayed in the sequence of ultrasound images;
wherein the time-ordered sequence of images comprises: a first sub-sequence of images displayed over a first time period, and a second sub-sequence of images displayed over a second time period;
wherein a first shadow pattern of the one or more shadow patterns is detected in the first sub-sequence of images and is not detected in the second sub-sequence of images; and
wherein the first shadow pattern is displayed or superimposed on the images in the second sub-sequence of images.

17. The method of claim 16, wherein the second time period is of such length that an operator can properly inspect the shadow pattern.

18. The method of claim 16, wherein the length of the second time period is adjustable by an operator.

19. The method of claim 16, wherein the first time period is so short that an operator cannot properly inspect the first shadow pattern.

20. The method of claim 16, further comprising:
displaying, in the second sub-sequence of images, a marker associated with the first shadow pattern and indicating a position of the first shadow pattern.

21. The method of claim 6, wherein image sticking of the detected shadow pattern comprises:
superimposing the detected shadow pattern on images in a subset of image frames of the time-ordered ultrasound images, wherein the shadow pattern was not detected in the subset of image frames of the time-ordered sequence of ultrasound image frames.
